(19) United States
(12) Patent Application Publication
(10) Pub. No.: US 2008/0132803 A1
(43) Pub. Date: Jun. 5, 2008

(54) METHOD AND SYSTEM FOR DOING BUSINESS BY MINING THE PLACENTAL-CHORD COMPLEX

(76) Inventor: Hyman Friedlander, Kiryat Ono (IL)

Correspondence Address: DR. MARK M. FRIEDMAN;C/O BILL POLKINGHORN - DISCOVERY DISPATCH, 9003 FLORIN WAY, UPPER MARLBORO, MD, 20772, US

(21) Appl. No.: 11/940,385

(22) Filed: Nov. 15, 2007

Related U.S. Application Data
(60) Provisional application No. 60/867,798, filed on Nov. 30, 2006.

(51) Int. Cl. A61B 10/00

(52) U.S. Cl. 600/562

(57) ABSTRACT

Methods, systems and computer readable code for mining the placenta-umbilical cord complex are disclosed. According to some embodiments, a plurality of distinct components of the placenta-umbilical complex are recovered for a placenta-umbilical cord complex of the donor and stored separately. Methods, systems and computer-readable code for determining which set of distinct components are recovered and stored, and how these components are allocated are disclosed. A method effecting a business transaction related to a placental-umbilical cord complex of a donor is disclosed were a transaction is effected whereby the donor donates a first set of components of the placental-chord complex, and the donor is provided with a private banking service for a second set of components of the placental-chord complex, wherein the first and second sets are distinct. Furthermore, methods systems and computer readable code for determining prices of transactions involving several distinct types of stem cells derived from the placenta-umbilical cord complex of a donor, or involving several different components of the placenta-umbilical the placenta-umbilical cord of a donor are disclosed. Furthermore, methods, systems and computer-readable code for maintaining a computer-based registry for different types of cells derived from the placenta-umbilical cord complex are disclosed.

### FIELD OF THE INVENTION

The present invention relates generally to methods, systems and computer-readable code for improving business performance and profitability, and particularly, the present invention relates to methods, systems and computer readable code for doing business by mining the placenta-umbilical cord complex.

### BACKGROUND AND RELATED ART

### Stem Cells From the Placental-Umbilical Cord Complex

As the modern understanding of disease has advanced, the potential utility of cell therapy for improving the prognosis of those afflicted has resulted in increased interest in new sources of human cells useful for therapeutic purposes. One such source of human cells is postpartum tissues, including the umbilical cord and the placenta, and fluids associated with the postpartum tissues, including umbilical cord blood and amniotic fluid.

Recently, attention has focused on the banking of umbilical cord blood (or simply “cord blood”) as a potential source of, for example, hematapoeitic cells for use by an individual for whom cord blood has been banked at birth. Such cells would be useful for those individuals, for example, who require therapeutic radiation which may eliminate functional portions of their immune system. Rather than requiring a bone marrow donor carefully matched to avoid rejection, the individual's own banked cord blood could be used to reconstitute the lost immune cells, and restore immune and hematological functions.

Still more recently, there has been interest in obtaining stem cells from cord blood, due to the wider potential therapeutic applications of such cells. Stem cells are understood in general terms as cells that 1) have the ability to self-renew for long periods through cell division from a single cell; and 2) have the ability to differentiate into specific cell types given the proper conditions. Since some populations of stem cells, and especially those from cord blood, do not require full match between donor and recipient, stem cells are potentially useful in treating a population of individuals, and not merely the person from whose cord blood the cells were initially obtained.

In particular, cord blood has been considered as a source of hematopoietic progenitor stem cells. Banked (or cryopreserved) cord blood, or stem cells isolated therefrom have been deemed useful for hematopoietic reconstitution, for example in bone marrow and related transplantations. (Boyse et al., U.S. Pat. Nos. 5,004,681 and 5,192,553).

The aforementioned banking of cord blood and stem cells therefrom has also been commercialized. In 2004 alone, a total of approximately 125,000 samples of cord blood stem cells were privately banked, an increase of 79% of the number in 2003. One example of an entity which provides a private stem cell banking service to expectant mothers whereby for a fee, umbilical cord blood is collected and cryopreserved for later use, is CorCell, Inc., which is headquartered in Philadelphia, Pa. Thus, there are private cord blood banks that provide the service for fee of preserving the stem cells for the new-borne and his family; and public cord blood banks that preserve cord blood unites for the public use, and they are usually not for profit operations.

It is noted that umbilical cord blood is not the only source of undifferentiated and partially differentiated cells in the placenta-umbilical cord complex. For example, other sources of therapeutic cells from the human umbilicus have been explored, including cells isolated from the Wharton's Jelly, umbilical vein or artery tissue, placenta and amniotic fluid.

For example, Purchio et al. (U.S. Pat. No. 5,919,702) have isolated chondrogenic progenitor cells (or prechondrocytes) from Wharton's Jelly. Mistry et al (U.S. patent application publication 2005/0054098) discloses methods of deriving from umbilical tissue isolated cells capable of self-renewal and expansion in culture. Weiss et al. (U.S. patent application publication 20054/0136967) discloses a method for obtaining stem cells from an umbilical cord matrix source substantially free of cord blood.

Not only may stem cells be derived from umbilical cord blood, umbilical cord matrix, and related tissues, but the placenta itself is also known as a source of stem cells. Thus, U.S. patent application publication 2005/0176139 discloses a method for obtaining and culturing stem cells from the post-partum placenta, e.g. the placenta which has been expunged from the uterus after birth and does not include the umbilical cord. These placental stem cells promise to be of equal and perhaps superior potential to umbilical cord blood stem cells.

Furthermore, it is noted that the amniotic fluid associated with the placenta-umbilical cord complex also contains stems cells which can be harvested and banked. US Patent Application 2005/0042595 discloses techniques for isolation and expansion of undifferentiated cells from amniotic fluid. Furthermore, this patent application notes that after cryopreservation, the revived cells were cultured and differentiated into various cell types, such as neural cells, adipogenic cells, and chondrogenic cells.

FIG. 1 enumerates some of the current and potential applications of stem cells derived from the placenta-umbilical cord complex. Although the number of benefits and potential benefits associated with stem cells derived from the placenta-umbilical cord complex is manifold, and although increasing public awareness of the benefits of preserving these stem cells is increasing, it is noted that to date only 5% of the population having babies are aware of these benefits.

Furthermore, among the population that is informed of the benefits of stern cell banking, only a certain subset is willing or able to pay for these banking services. Unfortunately, any decision to discard material of the placenta-umbilical cord complex without extracting and storing the potentially therapeutic stem cells, is irreversible. Furthermore, any decision to bank only cord blood stem cells, and to not recover (e.g. due to the cost) and store, for example Wharton's-jelly derived stem cells or placenta-derived stem cells is also irreversible.

Therefore, although the costs of preserving these stem cells has decreased over the past few years and is expected to decrease in the future, there is an ongoing need, even an urgent need, for methods and systems which reduce the cost of banking stem cells of the placenta-umbilical cord complex, thereby making these technologies available to a wider segment of the population.

Generic Biomaterials From the Placental-Umbilical Cord Complex

It is noted that the placenta-cord complex is a rich source for many other biologically important materials other than stem cells. Indeed, for many years, generic biomaterials such as placental proteins, hormones and other molecules were extracted and used, for example, in the cosmetic industry (e.g. for manufacturing advanced skin formulations or collagen for skin filling), the medical industry (e.g. use of materials derived from amniotic membranes for treating burns, e.g. use of materials for reconstructive surgery) and the pharmaceutical industry (e.g. use of enzymes derived form the placenta-cord complex for manufacturing vaccines).

A “generic” biomaterial is tissue or fluids (as opposed to a suspension of isolated cells) which can be batched from several placenta-umbilical cord complexes from different individuals and then used regardless to the genotype of the individual from which it was procured.

Over the past 15 years the rate of commercial utilization of generic products derived from the placenta has dropped dramatically, due to the rapid, worldwide onset of AIDS. Although it is possible, in theory, to test the donor from which the placenta sample is derived and to associate this data with the sample, the costs associated with this process in many cases exceeds the value of the placenta-derived commodity. Thus the human placenta is, in most cases, discarded despite the valuable materials the placenta provides. There is an ongoing need for methods and systems which enable economically feasible utilization of placenta, and which allows for the commercial utilization of products derived from placenta despite the need for AIDS testing.

### SUMMARY OF THE INVENTION

Embodiments of present invention are motivated by the observation that utilization of multiple components of the placenta-umbilical cord complex may reduce the cost of supplying each individual component to donors and/or the marketplace. Thus, in accordance with some embodiments of the present invention, specific combinations of components of the placenta-umbilical cord are harvested as separate components and then either sold, privately banked, donated to a public bank or any combination thereof.

By deriving economic value from a given combination of separate components, rather than relying on a single component, specific economic limitations associated the entrenched practice of harvesting single components are now overcome due to the presently disclosed economic synergy.

One surprising commercial result now disclosed is that many segments of the population, which to date can not or will not bear the costs associated with therapeutic treatments derived from privately banked stem cells, will now be granted access to various products and services derived from the placenta-umbilical cord complex. Thus, embodiments of the present invention provide affordable private banking of specific stem-cells useful in cell therapy based therapies to many who otherwise would not have been able to receive this care.

Furthermore, it is noted that the placenta-umbilical cord complex provides certain biomaterials (e.g. collagen, amniotic membranes) which are useful as raw materials in plastic surgery and in the cosmetic industry. According to current practice these biomaterials are discarded and not harvested, because the market value of these generic biomaterials is exceeded by the costs of testing the mothers and/or the babies to certify these components disease free. This problem is overcome by the present invention, wherein the harvesting of economically valuable combinations of the placenta-umbilical cord complex yields enough income to justify the cost of testing necessary to certify these biomaterials as disease-free.

It is now disclosed for the first time a method of processing biological matter of the placenta-umbilical complex of a mammalian donor, the method comprising the step of: a) receiving the placenta-umbilical cord complex of the donor, b) determining a set of at least two distinct components of the placenta-umbilical cord complex to recover and store, c) recovering the determined components derived from the placenta-umbilical cord complex of the donor; and d) separately storing each recovered component.

According to some embodiments, the entire (or substantially the entire) placenta-umbilical cord complex (e.g. all components, or substantially all components, of the embryonic sac) is received.

According to some embodiments, the stored components include at least two distinct samples of individual (e.g. individual “isolated” cells, such as suspended cells, as opposed to a tissue sample) undifferentiated or partially differentiated stem cells, wherein each respective sample is derived a different placenta-umbilical cord complex location.

Exemplary distinct locations include umbilical chord blood, the placenta, Wharton Jelly and amniotic fluid.

According to some embodiments, the stored components include at least two distinct samples of individual undifferentiated or partially differentiated stem cells, and each sample is of a different cell type, where the cell types are selected from the group consisting of mesenchymal stem cells, hematopoietic stem cells, endothelia progenitor cells, and epithelial progenitor cells.

According to some embodiments, a first component of the at least two distinct components is undifferentiated or partially differentiated cells and a second component of the at least two distinct components is a generic biomaterial.

Exemplary “generic biomaterial” is selected from the group consisting of vascular tissue (e.g. an umbilical chord blood vessel), extra-cellular matrix material (e.g., collagen, hyaluronic acid), cord blood plasma, membranes (e.g. amniotic membranes for burns), and enzymes.

According to some embodiments, the donor is a non-human donor, and the method further comprises designating at least two “veterinary” stored component for use as a therapeutic agent.

According to some embodiments, at least one stored component is sample of individual undifferentiated or partially differentiated cells, and the method further comprises the step of subjecting the undifferentiated or partially differentiated individual cells to an ex vivo expansion process and/or inducing differentiation of the individual cells ex vivo. It is noted that relevant methods of controlling proliferation and differentiation of stem and progenitor cells are disclosed in U.S. Pat. No. 6,962,698 of one of the present inventors and co-workers, though any relevant method of subjecting the undifferentiated or partially differentiated individuals cells to an ex vivo expansion process is within the scope of the present invention.

According to some embodiments, the mammalian donor or a family member (e.g. the mother) of the mammalian donor is pre-diagnosed with a genetic disease, at least one stored component is a sample of mesenchymal stem cells, and the method further comprises the step of designating the mesenchymal stem cells for use in screening a pharmaceutical composition related to the genetic disease.

According to some embodiments, at least one stored component is a sample of individual cells derived from the umbilical cord or the placenta, and the method further includes the step of designating the individual cells for use in a screening assay for medical product evaluation or for safety testing. In one example, the individual cells are offered for sale to a pharmaceutical company or another organization for screening a relevant product.

It is recognized that one or more of the harvested components have a “market value” and that this market value can vary from donor to donor. Furthermore, it is noted that the market value may fluctuate from time to time. Thus, according to some embodiments, the specific components selected to be harvested from the placenta-umbilical cord complex are selected in accordance with prevailing market conditions for components of the placenta-umbilical cord complex.

According to some embodiments, the determining is carried out in accordance with at least one of pricing data (for example, to maximize the price that harvested components might fetch on the market), demand data (for example, in accordance with a specific received order from a pharmaceutical company or a cosmetic concern), pricing forecasts, and demand forecasts of placenta-umbilical cord complex components.

One example of “pricing data” is the prevailing market prices of specific components of the placenta-umbilical cord complex. It is noted that as with any commodity, in some example, the “pricing data” may fluctuate as a function of time, and in many situations, it is one can forecast what future prices are and harvest components of the placenta-umbilical cord accordingly. In one example, price and/or demand forecasts are received via a computer data feed (e.g. a live feed).

Furthermore, it is also noted that the market values of the harvested components may vary from donor to donor. In one example, one or more pharmaceutical companies desire specific stem cells (for example, mesenchymal stem cells) from donors with donor type information (e.g. a certain genotype) and are willing to pay a premium for those cells. Those, accordance to this example, mesenchymal cells which may not have otherwise been harvested are specifically recovered and stored to supply this demand from the pharmaceutical companies.

In another example, a decision is made to privately bank mesenchymal stem cells of a donor who has an elevated risk of a neurological disease.

In another example, a decision is made to privately bank hematopoietic stem cells derived from the placenta-umbilical cord complex of a donor who has an elevated risk of a cancer (e.g. leukemia).

Furthermore, it is recognized that some donors are more likely to pay for private banking of more components of the placenta-umbilical cord complex than others, and thus, in some embodiments, the determining is carried out in accordance with economic data of the donor.

Exemplary “economic data” includes but is not limited to a sum of money the donor family (or the family's representative) is willing to pay, an economic status (e.g. annual income or other indicator of economic status) of the donor family, and an insurance status of the donor family.

According to some embodiments, the determining is carried out in accordance with at least one of a cost of testing the donor, a cost of testing a relative of the donor, and a cost of testing one or more components of the placenta-umbilical chord complex.

According to some embodiments, the presently disclosed method further includes the steps of e) designating a first set of stored components for private banking with a first business entity; and (f) offering a second set of stored components for sale to a second business entity, wherein the first set of components is distinct from the second set of components.

According to some embodiments, the first entity is a bank for biological matter (e.g. stem cells or plasma) and the second entity is one of a cosmetic industry entity and a pharmaceutical industry entity.

It is now disclosed for the first time a method of effecting a business transaction related to a placental-umbilical cord complex of a donor, where the placental-umbilical cord complex has a plurality of components. The presently disclosed method includes the steps of (a) effecting a transaction whereby the donor donates (e.g. makes publicly available where the donor forfeits the right to exclusive access, for example, by selling) a first set of components of the placental-chord complex and (b) providing to the donor a private banking service for a second set of components of the placental-chord complex, wherein the first and second sets are distinct.

According to some embodiments, the first set of donated components includes cord blood stem cells and the second set of privately banked components includes Wharton's Jelly derived cells.

According to some embodiments, the first set of donated components includes at least one generic biomaterial and said second set of privately banked components includes at least one sample of individual undifferentiated or partially differentiated cells.

According to some embodiments, the generic biomaterial is selected from the group consisting of vascular tissue (e.g. an umbilical chord blood vessel), extra-cellular matrix material (e.g., collagen, hyaluronic acid), cord blood plasma, membranes (e.g. amniotic membranes which are useful, for example, for treating burns), and enzymes (e.g. hyaluronidase).

According to some embodiments, a decision of which the components are to be donated and which said components are to be privately banked is carried out in accordance with at least one of a medical data of the donor and economic data of the donor, and economic demand data (e.g. received orders, pricing data) of the components of said placenta-umbilical cord complex.

According to some embodiments, the method further includes the step of computing a price of the transaction associated with said donating and said banking.

It is noted that in some examples, the “price” of the transaction may reflect monies required by the donor or a representative of the donor to effect the transaction related to one or more components of the placenta-umbilical cord complex. Alternatively, the “price” of the transaction may reflect monies paid to the donor for effecting the transaction related to one or more components of the placenta-umbilical cord complex.

Furthermore, it is noted that in many examples, a representative of the donor family is required to sign a formal legal contract for the transaction (e.g. a transaction which includes private banking, donation of components, or a combination thereof. Certain embodiments of the present invention provide computer implemented methods and/or computerized systems operative to automatically generate the appropriate formal legal contract text for consummating the transaction, where the contract reflects which components are to be privately banked, which components are to be donated, and required payments or monies to be received by a representative of the donor. Thus, according to some embodiments of the present invention, the method further includes the step of generating formalized contract text describing a transaction associated with the donating and the banking.

It is now disclosed for the first time a method of determining a price of a financial transaction involving several distinct types of stem cells derived from the placenta-umbilical cord complex of a donor. The presently method methods includes the steps (a) determining a cost of privately banking first set of samples of stem cells derived from the placenta-umbilical cord complex of the donor, (b) determining a market value of a second set of samples of stem cells derived from the placenta-umbilical cord complex of the donor, such that the first set and said second set of cells have different stem cell type profiles, and (c) determining the price of the stem cell transaction by computing a function of the cost of the private banking of the first set of stem cells and the market value of the second set of stem cells.

According to some embodiments, the price of the stem cell transaction is further determined in accordance with costs testing at least one of the donor and a family member of the donor.

According to some embodiments, the price of the stem cell transaction is determined in accordance with donor type information (e.g. medical history of the patient, donor genotype). In one example, a pharmaceutical company desires stem cells of a donor's genotype for screening assays, and is willing to pay a premium for these stem cells. According to this example, the determined or calculated price of the transaction is computed in accordance with this premium.

According to some embodiments, the method further includes the step of determining a market value of one or more generic biomaterials derived from the placenta-umbilical cord complex of the donor, wherein the determining of the price of stem cell transaction is carried out in accordance with the market value of the one or more generic biomaterials.

According to some embodiments, the market value of the second set of samples is determined in accordance with at least one of a medical history of the donor, a medical history of a family member of the donor and a genetic profile of the donor.

It is now disclosed for the first time a method of maintaining a computer-based registry different types of undifferentiated or partially differentiated cells derived from different locations in the placenta-umbilical cord complex. The presently disclosed method includes the steps of (a) creating a new donor record for a potential donor in a placenta-cord complex cells database of the registry, (b) storing donor identification information in the new record, (c) storing sample set identification information in the new record, the sample set including a plurality of samples of distinct stem cell types of the placenta-umbilical cord complex, (d) collecting the sample set from the donor, (e) obtaining donor type information and storing the donor type information in the new record; (f) storing an availability indication with the new record to indicate which stem cell types are available for public use, and (g) storing the collected sample set in a bank such that individual samples of distinct stem cell types can be obtained from the bank using the stored sample set identification information; and (h) modifying the availability indication for a particular donor record when the availability for public use of at least one type of stem cells changes.

Thus, it is noted that at a point in time after the time of initial storage of the stem cells, the designation of which stem cells types are publicly available and which stem cell types are not publicly available (e.g. privately banked) can change. According to one example, a family member of a donor exhibits an elevated risk of neurological disease at the time of birth. As such, it is decided to privately bank (e.g. make these cells unavailable for public usage) mesenchymal stem cells in case the family member is in need of a stem cell transplant. At a later time, the family member is no longer in need of the mesenchymal stem cells that were privately banked (for example, the family member receives appropriate treatment from another source, or passes away), and it is decided to make only mesenchymal stem cells available for public use while maintaining the “privately banked” status of other types of stem cells, such as hemaptopoietic stem cells. Thus, according to this example, the availability indication stored in the database and associated with the mesenchymal stem cells would be modified to indicate that these mesenchymal stem cells of the donor are now publicly available.

It is now disclosed for the first time a method of utilizing the placenta-umbilical cord complex of a donor. The presently disclosed method methods includes the steps of (a) testing for disease at least one of the donor and a relative of the donor, and (b) if results of the testing indicates a disease free state, (c) privately banking stem cells of the placenta-umbilical cord complex; and (d) offering for sale generic biomaterials of the placenta-umbilical cord comnplex.

It is noted that today, many generic biomaterials are not harvested from the placenta-umbilical cord complex, because the market value of these biomaterials is exceeded by the cost of testing. Embodiments of the present invention overcome this limitation, because donors are induced to pay for testing in order to privately bank stem cells of the placenta-umbilical cord complex. Thus, according to some embodiments, test results certifying the generic biomaterials as disease-free are transferred to the organization or corporation which purchases the genetic biomaterials, allowing for their use. Furthermore, because a payment is received for these generic biomaterials, the donor's stem cells may be banked at a reduced price.

In one example, a family of pregnant women is offered to privately store for future use a variety of components, such as cord blood stem cells, Wharton's Jelly-derived cells, plasma or platelet rich plasma, placenta-derived MSCs, blood vessels derived epithelial progenitors; and any combination of those.

An additional embodiment includes the utilization of the umbilical cord MSC, derived from Wharton's Jelly or from other umbilical cord sources, to be used in a three dimensional matrix together with media containing cytokines and growth factors for the in vitro expansion of umbilical cord blood. According to some examples in accordance with this embodiment, the MSC are used as supporting matrix for growing hematopoietic stem cells.

These and further embodiments will be apparent from the detailed description and examples that follow.

### BRIEF DESCRIPTION OF THE DRAWINGS

FIG. 1 provides a diagram of current and potential applications of stem cells.

FIGS. 2 and 4 provides a block diagram of a computerized system in accordance with some embodiments of the present invention.

FIG. 3 provides a flow chart of a method for calculating a parameter in accordance with some embodiments of the present invention.

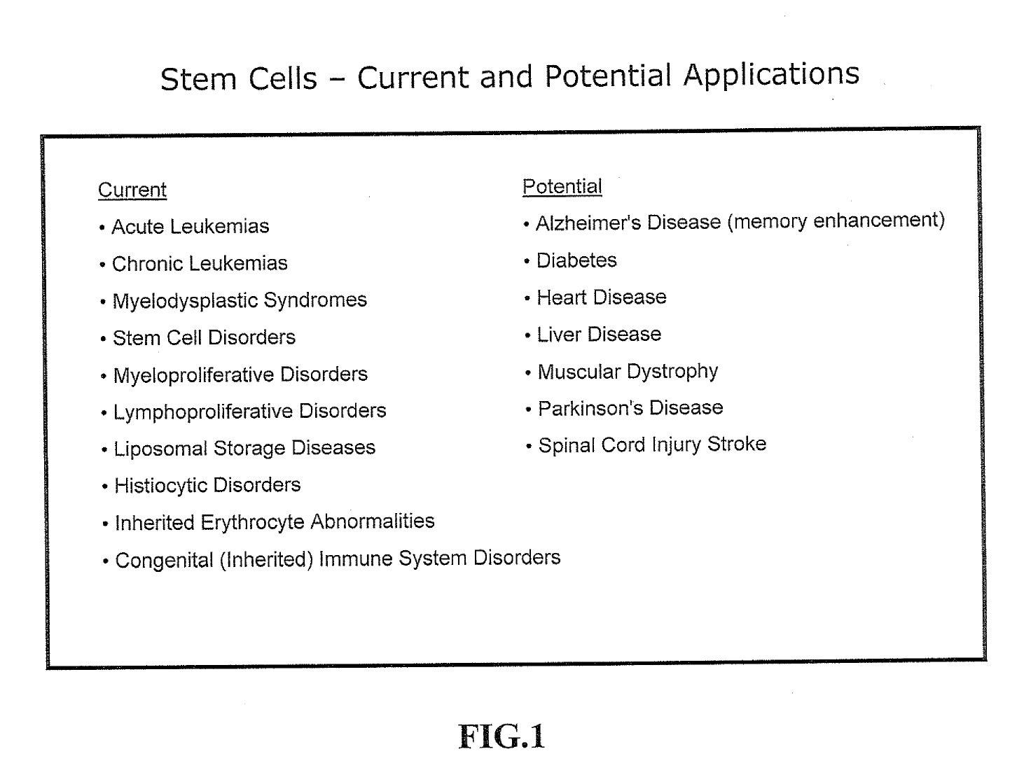

### DETAILED DESCRIPTION OF THE INVENTION

### Definitions

Various terms used throughout the specification and claims are defined as set forth below.

Stem cells are undifferentiated cells defined by the ability of a single cell both to self-renew, and to differentiate to produce progeny cells, including self-renewing progenitors, non-renewing progenitors, and terminally differentiated cells. Stem cells are also characterized by their ability to differentiate in vitro into functional cells of various cell lineages from multiple germ layers (endoderm, mesoderm and ectoderm), as well as to give rise to tissues of multiple germ layers following transplantation, and to contribute substantially to most, if not all, tissues following injection into blastocysts.

Stem cells are classified according to their developmental potential as: (1) totipotent; (2) pluripotent; (3) multipotent; (4) oligopotent; and (5) unipotent. Totipotent cells are able to give rise to all embryonic and extraembryonic cell types. Pluripotent cells are able to give rise to all embryonic cell types. Multipotent cells include those able to give rise to a subset of cell lineages, but all within a particular tissue, organ, or physiological system (for example, hematopoietic stem cells (HSC) can produce progeny that include HSC (self-renewal), blood cell-restricted oligopotent progenitors, and all cell types and elements (e.g., platelets) that are normal components of the blood). Cells that are oligopotent can give rise to a more restricted subset of cell lineages than multipotent stem cells; and cells that are unipotent are able to give rise to a single cell lineage (e.g., spermatogenic stem cells).

Stem cells are also categorized on the basis of the source from which they may be obtained. An adult stem cell is generally a multipotent undifferentiated cell found in tissue comprising multiple differentiated cell types. The adult stem cell can renew itself. Under normal circumstances, it can also differentiate to yield the specialized cell types of the tissue from which it originated, and possibly other tissue types. An embryonic stem cell is a pluripotent cell from the inner cell mass of a blastocyst-stage embryo. A fetal stem cell is one that originates from fetal tissues or membranes. A postpartum stem cell is a multipotent or pluripotent cell that originates substantially from extraembryonic tissue available after birth, namely, the placenta and the umbilical cord. These cells have been found to possess features characteristic of pluripotent stem cells, including rapid proliferation and the potential for differentiation into many cell lineages. Postpartum stem cells may be blood-derived (e.g., as are those obtained from umbilical cord blood) or non-blood-derived (e.g., as obtained from the non-blood tissues of the umbilical cord and placenta).

A mesynchymal, placental, cord blood, or other stem cell may be characterized by its cell markers. A variety of cell markers are known. See e.g., Stem Cells: Scientific Progress and Future Research Directions. Department of Health and Human Services. June 2001. http://www.nih.gov/news/stemcell/scireport.htm. Cell markers may be detected by methods known in the art, such as by immunochemistry or flow cytometry. Flow cytometry allows the rapid measurement of light scatter and fluorescence emission produced by suitably illuminated cells or particles. The cells or particles produce signals when they pass individually through a beam of light. Each particle or cell is measured separately and the output represents cumulative individual cytometric characteristics. Antibodies specific to a cell marker may be labeled with a fluorochrome so that it may be detected by the flow cytometer. See, eg., Bonner et al., Rev. Sci. Instrum 43: 404-409, 1972; Herzenberg et al., Immunol. Today 21: 383-390, 2000; Julius et al., PNAS 69: 1934-1938, 1972; Ormerod (ed.), Flow Cytometry: A Practical Approach, Oxford Univ. Press, 1997; Jaroszeski et al. (eds.), Flow Cytometry Protocols in Methods in Molecular Biology No. 91, Humana Press, 1997; Practical Flow Cytometry, 3^{rd} ed., Wiley-Liss, 1995.

Embodiments of the present invention refer to utilization of the “placenta-umbilical cord complex.” As used herein, the placenta-umbilical cord complex includes the post-partum placenta, the post-partum umbilical cord (e.g. umbilical cord vasculature, blood and the umbilical cord matrix or Wharton's Jelly), and associated fluids and tissues (e.g. amniotic fluid and amnion). The placenta-umbilical cord complex may be obtained from any mammalian species, including rodents, human, non-human primates, equines, canines, felines, bovines, porcines, ovines, lagomorphs, and the like. In an embodiment of the invention, the placenta-umbilical cord complex is obtained from human.

It is noted that the placenta-umbilical cord complex includes a plurality of “components” including different types of stem cells, where each type of stem cell can be considered a different compound, biomaterials (e.g. blood vessels, extracellular matrices), and fluids associated with the placenta-cord complex (e.g. cord blood or amniotic fluid). As used herein, “separately storing” components of the placenta-umbilical cord complex implies that these components first separated from each other during or after harvesting, and thus sorted before storing.

As used herein, “mining” the placenta-umbilical cord complex includes obtaining a plurality of components from the placenta-umbilical cord complex and storing, selling or utilizing each component and any combination of two or more components.

Embryonic tissue is typically defined as tissue originating from the embryo (which in humans refers to the period from fertilization to about six weeks of development. Fetal tissue refers to tissue originating from the fetus, which in humans refers to the period from about six weeks of development to parturition. Extraembryonic tissue is tissue associated with, but not originating from, the embryo or fetus. Extraembryonic tissues include extraembryonic membranes (chorion, amnion, yolk sac and allantois), umbilical cord and placenta (which itself forms from the chorion and the maternal decidua basalis).

As used herein, a “set of samples” of stem cells is one or more samples of stem cells.

As used herein, a stem cell “type” relates to either the source from where the stem cell is obtained (e.g. from the cord blood, from the Wharton's jelly, from the placenta, or from the amniotic fluid) or to the biological characteristics of the stem cell (e.g.). Thus, in one example, mesenchymal stem cells from the cord blood and from the umbilical cord matrix or Wharton's jelly are to be considered different kinds of stem cells. In one example stem cells for hematopoietic and/or immune tissues and mesenchymal stem cells, both harvested from the cord blood, are to be considered different types of stem cells.

As used in, a “stem cell type profile” defines the distribution of quantities of stem cells types in a set of samples, or the ratios between the quantities of stem cell types in a set of samples. Thus, according to one particular example, where all stem cell sachets are the same size, a first set of samples containing 2 sachets of mesenchymal stem cells and 10 sachets of cord blood stem cells has a “stem cell type profile” which differs from a second set of samples containing 4 sachets of mesenchymal stem cells and 1 sachets of cord blood stem cells.

The “donor type information” includes information related to the donor or the donor's family. Thus, the donor type information at least one of genetic type information, donor phenotype, family medical history and information relating to ethnic and geographic origin of the donor. Other donor information such as donor phenotype may also be included.

As used herein, a “price of a transaction” or a “price of a placenta-umbilical cord complex transaction” includes an amount of money to be collected from a donor or his representative, or a payout to the donor or his representative for at least one of the banking of biological matter derived from the donor's placenta-umbilical cord complex and the donation or sale of biological matter derived from the donor's placenta-umbilical cord complex.

Differentiation is the process by which an unspecialized (“uncommitted”) or less specialized cell acquires the features of a specialized cell, such as a nerve cell or a muscle cell, for example. A differentiated cell is one that has taken on a more specialized (“committed”) position within the lineage of a cell. The term committed, when applied to the process of differentiation, refers to a cell that has proceeded in the differentiation pathway to a point where, under normal circumstances, it will continue to differentiate into a specific cell type or subset of cell types, and cannot, under normal circumstances, differentiate into a different cell type or revert to a less differentiated cell type. De-differentiation refers to the process by which a cell reverts to a less specialized (or committed) position within the lineage of a cell. As used herein, the lineage of a cell defines the heredity of the cell, i.e. which cells it came from and what cells it can give rise to. The lineage of a cell places the cell within a hereditary scheme of development and differentiation.

The stem cells derived from the umbilical cord-placenta complex of the invention may also be cryopreserved. Methods for cryopreserving cells are well known in the art, and any acceptable method is within the scope of the present invention. For example, the cells may be cryopreserved in a solution comprising, for example, dimethyl sulfoxide at a final concentration not exceeding 10%. The cells may also be cryopreserved in a solution comprising dimethyl sulfoxide and/or dextran. Other methods of cryopreserving cells are known in the art.

In a broad sense, a progenitor cell is a cell that has the capacity to create progeny that are more differentiated than itself, and yet retains the capacity to replenish the pool of progenitors. By that definition, stem cells themselves are also progenitor cells, as are the more immediate precursors to terminally differentiated cells. When referring to the cells of the present invention, as described in greater detail below, this broad definition of progenitor cell may be used. In a narrower sense, a progenitor cell is often defined as a cell that is intermediate in the differentiation pathway, i.e., it arises from a stem cell and is intermediate in the production of a mature cell type or subset of cell types. This type of progenitor cell is generally not able to self-renew. Accordingly, if this type of cell is referred to herein, it will be referred to as a non-renewing progenitor cell or as an intermediate progenitor or precursor cell.

As used herein, the phrase differentiates into a mesodermal, ectodermal or endodermal lineage refers to a cell that becomes committed to a specific mesodermal, ectodermal or endodermal lineage, respectively. Examples of cells that differentiate into a mesodermal lineage or give rise to specific mesodermal cells include, but are not limited to, cells that are adipogenic, chondrogenic, cardiogenic, dermatogenic, hematopoetic, hemangiogenic, myogenic, nephrogenic, urogenitogenic, osteogenic, pericardiogenic, or stromal. Examples of cells that differentiate into ectodermal lineage include, but are not limited to epidermal cells, neurogenic cells, and neurogliagenic cells Examples of cells that differentiate into endodermal lineage include, but are not limited to, pleurigenic cells, hepatogenic cells, cells that give rise to the lining of the intestine, and cells that give rise to pancreogenic and splanchogenic cells.

It is noted that stem cells derived from the placenta-umbilical cord complex may be used in the treatment of any kind of injury due to trauma where tissues need to be replaced or regenerated. Examples of such trauma-related conditions include central nervous system (CNS) injuries, including injuries to the brain, spinal cord, or tissue surrounding the CNS injuries to the peripheral nervous system (PNS), or injuries to any other part of the body. Such trauma may be caused by accident, or may be a normal or abnormal outcome of a medical procedure such as surgery or angioplasty. The trauma may be related to a rupture or occlusion of a blood vessel, for example, in stroke or phlebitis. In specific embodiments, the cells may be used in autologous or allogeneic tissue replacement or regeneration therapies or protocols, including, but not limited to treatment of corneal epithelial defects, cartilage repair, facial dermabrasion, mucosal membranes, tympanic membranes, intestinal linings, neurological structures (e.g., retina, auditory neurons in basilar membrane, olfactory neurons in olfactory epithelium), burn and wound repair for traumatic injuries of the skin, or for reconstruction of other damaged or diseased organs or tissues. Injuries may be due to specific conditions and disorders including, but not limited to, myocardial infarction, seizure disorder, multiple sclerosis, stroke, hypotension, cardiac arrest, ischemia, inflammation, age-related loss of cognitive function, radiation damage, cerebral palsy, neurodegenerative disease, Alzheimer's disease, Parkinson's disease, Leigh disease, AIDS dementia, memory loss, amyotrophic lateral sclerosis (ALS), ischemic renal disease, brain or spinal cord trauma, heart-lung bypass, glaucoma, retinal ischemia, retinal trauma, inborn errors of metabolism, adrenoleukodystrophy, cystic fibrosis, glycogen storage disease, hypothyroidism, sickle cell anemia, Pearson syndrome, Pompe's disease, phenylketonuria (PKU), porphyrias, maple syrup urine disease, homocystinuria, mucoplysaccharide nosis, chronic granulomatous disease and tyrosinemia, Tay-Sachs disease, cancer, tumors or other pathological or neoplastic conditions.

### DESCRIPTION

The placenta-umbilical cord complex provides several types of cells and biological materials that may be utilized as therapeutic agents for both human and veterinary clinical applications as well as source for raw materials for various medical, scientific and cosmetic products. The present inventors are disclosing for the first time a method wherein various combinations of some or all of the following resources from the placenta-umbilical cord of a donor are extracted, stored, and utilized:

1) umbilical cord blood

2) Wharton's Jelly or umbilical cord matrix.

3) blood vessels of the umbilical cord or the placenta.

4) the placenta

5) amniotic fluid.

It is noted that although techniques for utilizing any single component of the aforementioned list of five components are known in the art, the entrenched practice is to extract a single type of desired cells, material or compound from a single component and to discard what remains of the placenta-umbilical cord complex. The present invention provides business methods for utilizing combinations of these resources in order to reduce the costs associated with harvesting and utilizing each resource.

### Umbilical Cord Blood (Component #1)

The umbilical cord blood provides:

a) samples of specific types of cells, such as stem and progenitor cells for hematopoietic and immune tissues, or mesenchymal stem cells and other types of stem cells have been reported to be found in umbilical cord blood (Erices A, Conget P, Minguell JJ. Mesenchymal progenitor cells in human umbilical cord blood. Br J Haematol 2000; 109:235-42).

b) platelet rich cord blood derived plasma, which is useful for cell culture techniques including the growing of cells in autologous conditions or as a component a freezing medium within minimum use of foreign protein.

It is noted that the recovery and sale of the cord blood derived plasma provides income which can, in some embodiments, defer the cost of banking the stem cells.

### Wharton's Jelly (Component #2)

Wharton's Jelly—umbilical cord matrix or Wharton's Jelly is known as a source of mesenchymal stem cells and other types of stem and/or progenitor cells as disclosed in US Patent Application publication 20040136967 and US Patent Application publication US 2005/0054098.

Furthermore, it is noted that the umbilical cord matrix contains hyaluronic acid and collagen, which can be extracted and sold for use as generic biomaterial for the cosmetic industry. It is noted that income from the sale of the hyaluronic acid may defer the costs of providing private banking services to the donor.

### Umbilical Cord Blood Vessels (Component #3)

It is noted that human umbilical cord blood vessels are used as surgical grafts, for example, see (Mamode N, Scott R N, Graft type for femoro-popliteal bypass surgery, The Cochrane Database of Systematic Reviews 2005 Issue 4).

(see Daniel J. et al Development of the human umbilical vein scaffold for cardiovascular tissue engineering applications ASAIO J. 2005 May-June; 51(3):252-61)

Thus, according to one example, a donor donates the umbilical cord blood vessels, and at least a portion of the income received from the donated umbilical cord blood vessels helps to defer a portion of the cost of privately banking stem cells. This reduces the cost of banking the stem cells.

### Placenta (Component #4)

The placenta is a source of placenta stem cells, and it is noted that US Patent Application publication 2005/0176139 discloses methods of obtaining and culturing stem cells from the placenta (e.g. the post partum placenta that has been expunched from the uterus after birth and does not include the umbilical cord). The placenta is also used as a source for nutritional factors and other materials for the cosmetic industry as well as a support for ex vivo growth of cells.

It is noted that the placenta is also a source of collagen. It is noted that the present invention provides a business method whereby the testing cost of certifying the placenta as disease free is defrayed by harvesting other components of the placenta-umbilical cord complex, or is defrayed by monies collected by the donor for private banking services. Thus, in accordance with some embodiments of the present invention, the presently disclosed business methods allow for the commercialization of a process for extracting tested disease free collagen (and other useful proteins) from placenta.

### Amniotic Fluid (Component #5)

The amniotic fluid is also a source of stem cells. US Patent Application publication 2005/0042595 discloses a cell banking system including a plurality of preserved, viable samples containing multipotent amniotic fluid-derived cells. The amnion is also a source for stem cells.

### Exploitation of Harvested Stem Cells

Techniques for banking stem cells are well known in the art. Furthermore, the banking of cord cells stem cells has been commercialized for years by various companies (for example, see U.S. Pat. No. 5,993,387). In general, there are two kinds of cord stem cell banks. The first kind of cord stem cell banks, family banks or “private” banks, store harvested cord stem cells for a donor's family and provide a sample of the donated cord stem cells back to the donor family if needed.

The second type of bank, generally referred to as “public banks” have been established to provide typed, anonymous samples to the general public based on genetic matching with needy potential recipients. A general discussion of various ethical issues relating to cord-blood banks is provided in Jeremy Sugarman et al, “Ethical Aspects of Banking Placental Blood for Transplantation,” 274 JAMA 22, pp. 1783-85, Dec., 13, 1995.

In general, stem cells derived form the placenta-umbilical cord complex (e.g. from cord blood, or Wharton's jelly or the placenta or the amniotic fluid) can be privately banked or publicly banked. The therapeutic value of stem cell transplants are well known, and in typically when stem cells are banked type information is stored in a database to deliver the stem cells to a recipient who is compatible with the stem cells.

Alternatively or additionally, the stored stem cells can be sold for use in research. In one example, the stem cells derived from the placenta, from umbilical cord blood, or from amniotic fluid may be used in a functional assay for medical product safety or for screening of drugs, or for pharamacogenomics, or for targeted drug design In one example, the income obtained from selling one or more types of stem cells for use in research helps to defray the costs associated with privately banking certain types of stem cells.

### Computerized Systems, Computer Implemented Methods and Computer Readable Code

Some embodiments of the present invention related to computerized systems, computer implemented methods and computer readable code. In particular, some embodiments of the present invention relate to data management systems and/or computational systems and/or decision support systems.

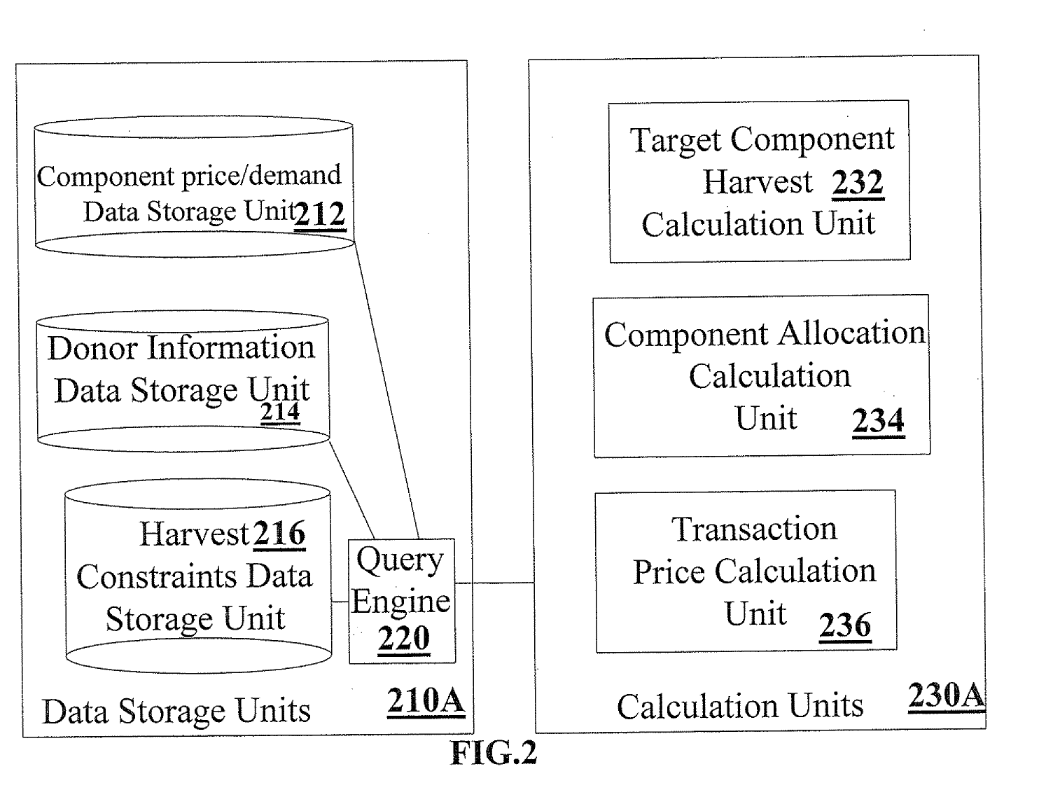

FIG. 2 provides a block diagram of an exemplary decision support system for calculating various parameters related to mining and storing components of the placenta-umbilical cord complex according to some embodiments of the present invention. In general, the system includes one or more of data storage units 210A which provide data to one or more calculation units 230. Relevant data from each data unit may be accessed through an optional query engine 220. In some embodiments, the query engine is also linked to a user interface (not shown), and human users may access data directly from the query engine.

In general, it is desired, before a placenta-umbilical cord is harvested to determine one or more relevant parameters. For example, in many situations, the same placenta-umbilical cord complex components are not necessarily harvested from each donor, and not necessarily in the same ratios and the same quantities. Instead, specific components or quantities of components are harvested for specific donors or groups of donors. In one example, certain components which are not normally harvested may be harvested from a donor having a certain genotype. The target component harvest calculation unit 232 is operative to determine which components are to be harvested.

This determination is carried out in accordance with one or more data sets provided by the data storage units 210A. Thus, each “data storage unit” is operative to store a relevant data set in volatile or non-volatile memory, while the “query engine” 220 is operative to provide access to the data. Exemplary data storage units include but are not limited to a components price or demand data storage unit 212, a donor information data storage unit 214, and a harvest constraints data storage unit 216.

The component price or demand storage unit 212 may provide the current price and/or future anticipated price of any component or combination of components of the placenta-umbilical cord complex 212. In some embodiments, one or more data storage units is supplied with data via a data feed. It is noted that in many examples there is a need or desire to supply a certain component to a certain person or entity where a “price” is not necessarily paid, but some sort of goodwill is generated (e.g. community benefit), and in some embodiments, these considerations may be weighted (and scored) when performing various calculations.

Although the data component price/demand data storage unit 212 and the donor information data storage unit 214 are illustrated separately, in some embodiments they are operatively linked to each other. Thus, in one example, the demand and/or price for certain components of the placenta-umbilical cord complex depends, for example, on the genotype of the donor.

It is noted that many useful parameters may be determined on the basis of these data. Thus, in one example, it is desired to maximize the economic value of the components harvested from the placenta-umbilical cord complex. Towards this ends, a decision needs to be taken about specifically which components will be harvested and optionally how much of each component (e.g. the target components to be harvested). The target component harvest calculation unit 232 determines what the prevailing prices are of each component, and after comparing the “value” of each possible combination (for example, by using a scoring function) selects the highest scoring combination.

It is noted that this highest scoring combination may depend on a number of factors including the prevailing or expected demand or market price of various components (which may also depend, for example, on the locale of the donor), donor information (e.g. genotype of the donor or the economic situation of the donor) and “harvest constraints.”

It is recognized that when harvesting various components of the placenta-umbilical cord complex that there situations where harvesting one component of the placenta-umbilical cord complex would render another component not harvestable. In this case, a choice may need to be made about what to harvest and what not to harvest. Thus, there may be certain “constraints” that need to be considered about what components to harvest, and certain desired combinations of components may not be considered harvestable. It is noted these “harvest constraints” (e.g. combinations of components that may not be harvested for various reasons) are not necessarily static, and may dependent on location (e.g. hospital, etc) as well as technology. Thus, in some embodiments, any one of the data storage units including the harvest constraints data storage unit may be updated.

Furthermore, it is recognized that certain harvested components have more than one application and may be utilized in more than one manner. Furthermore, it is noted that how a component is utilized or should be utilized may also depend on economic factors or medical data related to the donor which is provided by donor information data storage unit 214. Thus, in some embodiments, a component allocation calculation unit 234 is provided to determine (e.g. based on a scoring function) how a component will be used or marked (e.g. for private banking or for public banking or for some type of semi-public banking) or to which destination a component will be sent after harvest. In some embodiments, the target components to be harvested and the allocation parameters are inter-related and thus, the target component harvest unit 232 may be operatively linked to the component allocation calculation unit 234. In one example, the target components and the component allocations are determined together, for example, using a scoring function.

In some embodiments, a transaction price calculation unit 236 is provided to determine a cost of a transaction 236. This will allow a determination of how much money should be collected from the donor or paid to the donor.

In one example, one or more parameters determined by a calculation unit is provided to a potential donor, for example, over the Internet. This will allow brokers of placenta-umbilical cord complexes to market their services and present to potential donors a variety of options for donating and/or banking certain components of the placenta-umbilical cord complex. Thus, in one example, a user will be asked to provide his or her medical and/or economic data and will be present with possible packages that include private and public banking. This e-commerce embodiment may help a potential donor decide in advance which transaction is best for him or her.

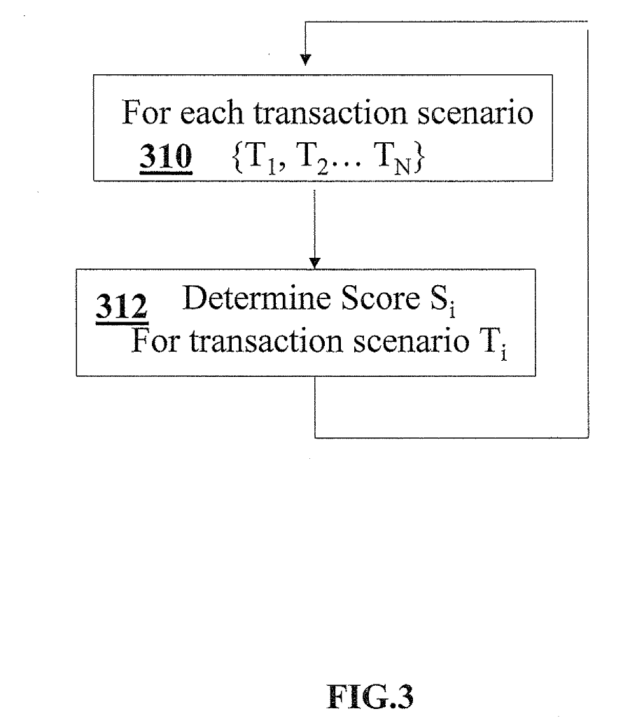

FIG. 3 provides a flow chart of a certain exemplary procedure for calculating an optimal set of components to harvest and/or an optimal allocation or components. Thus, for a given placenta-umbilical cord complex, a variety of different options are examined, each of which is referred to as a transaction scenario. For the case of target components to be harvested, these options may include different combinations of components. Similarly, a plurality of allocation scenarios may be examined. Furthermore, in some examples, combinations of harvest scenarios and allocation scenarios may be examined.

Each scenario 310 is then scored 312 in accordance with data provided by one or more data storage units 210A, and the scenario with the highest score is selected.

It is noted that decisions about allocations of distinct components of the placenta-umbilical cord complex may be made after the components are harvested. In one example, a certain set of components are publicly banked and others are privately banked. At a later date, the price or demand of a banked components may fluctuate, and/or the donor's economic situation or medical situation may change. Thus, it is possible that a decision may need to be made at a later date about how to change the allocation of various distinct components of the placenta-umbilical cord complex. There is a need to calculate a price of a transaction associated with this decision.

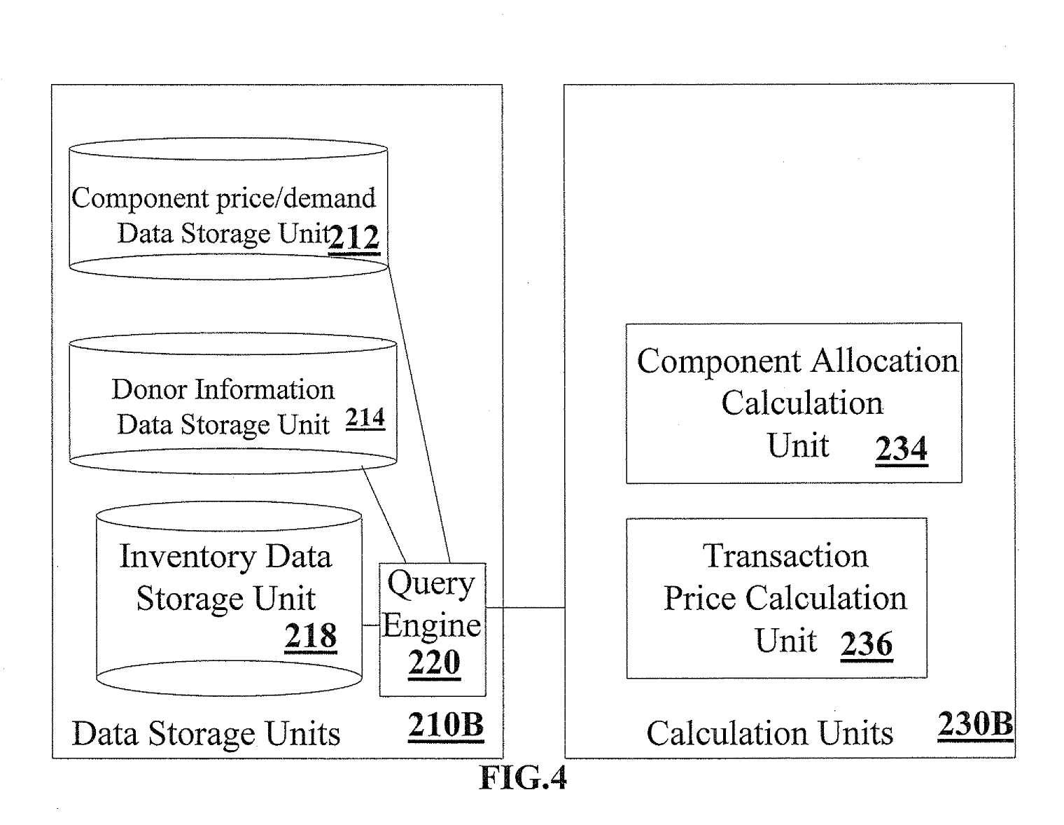

FIG. 4 provides a block diagram of an exemplary decision support system for calculating how to re-allocate components at a time after the placenta-umbilical cord is initially harvested. The exemplary system includes an inventory data storage unit 218 for providing data about the current status of various stored components of the placenta-umbilical cord complex. The component allocation calculation unit 234 determines an optimal manner in which to reallocate stored components of the placenta-umbilical cord complex (for example, by choosing a scenario with the highest score). The transaction price calculation unit 236 calculates a cost or price associated with the re-allocation of various placenta-umbilical cord complex components.

In the description and claims of the present application, each of the verbs, “comprise” “include” and “have”, and conjugates thereof, are used to indicate that the object or objects of the verb are not necessarily a complete listing of members, components, elements or parts of the subject or subjects of the verb.

The present invention has been described using detailed descriptions of embodiments thereof that are provided by way of example and are not intended to limit the scope of the invention. The described embodiments comprise different features, not all of which are required in all embodiments of the invention. Some embodiments of the present invention utilize only some of the features or possible combinations of the features. Variations of embodiments of the present invention that are described and embodiments of the present invention comprising different combinations of features noted in the described embodiments will occur to persons of the art.

What is claimed is:

1. A method of processing biological matter of the placenta-umbilical complex of a mammalian donor, the method comprising
a) receiving the placenta-umbilical cord complex of the donor,
b) determining a set of at least two distinct components of the placenta-umbilical cord complex to recover and store;
c) recovering said determined components derived from the placenta-umbilical cord complex of the donor; and
d) separately storing each said recovered component.

2. The method of claim 1 wherein said stored components include at least two distinct samples of individual undifferentiated or partially differentiated stem cells, each respective said sample derived a different placenta-umbilical cord complex location.

3. The method of claim 2 wherein said distinct locations are selected from the group consisting of umbilical chord blood, placenta, Wharton Jelly and amniotic fluid.

4. The method of claim 1 wherein said stored components include at least two distinct samples of individual undifferentiated or partially differentiated stem cells, each said sample of a different type, and said cell types are selected from the group consisting of mesenchymal stem cells, hematopoietic stem cells, endothelia progenitor cells, and epithelial progenitor cells.

5. The method of claim 1 wherein a first said component is undifferentiated or partially differentiated cells and a second said component is a generic biomaterial.

6. The method of claim 4 wherein said generic biomaterial is selected from the group consisting of vascular tissue, extra-cellular matrix material cord blood plasma, and membranes.

7. The method of claim 1 wherein said donor is a non-human donor, the method further comprising:
e) designating at least one said stored component for use as a therapeutic agent.

8. The method of claim 1 wherein at least one said stored component is sample of individual undifferentiated or partially differentiated cells, the method farther comprising:
e) performing at least one of subjecting said undifferentiated or partially differentiated individual cells to an ex vivo expansion process and inducing differentiation of the individual cells ex vivo

9. The method of claim 1 wherein the mammalian donor or a mother of the mammalian donor is pre-diagnosed with a genetic disease, at least one said stored component is a sample of mesenchymal stem cells, the method further comprising:
e) designating said mesenchymal stem cells for use in screening a pharmaceutical composition related to said genetic disease.

10. The method of claim 1 wherein at least one said stored component is a sample of individual cells derived from the umbilical cord or the placenta, the method further comprising:
e) designating said individual cells for use in a screening assay for medical product evaluation

11. The method of claim 1 wherein said determining is carried out in accordance with at least one of pricing data, demand data, pricing forecasts, and demand forecasts of placenta-umbilical cord complex components.

12. The method of claim 1 wherein said determining is carried out in accordance with donor type information.

13. The method of claim 12 wherein said determining includes making a determination to store mesenchymal stem cells if said donor type information indicates an elevated risk of a neurological disease.

14. The method of claim 12 wherein said determining includes making a determination to store hematopoietic stem cells if said donor type information indicates an elevated risk of a cancer.

15. The method of claim 1 wherein said determining is carried out in accordance with economic data of the donor.

16. The method of claim 15 wherein said economic data includes at least one of sum of money the donor is willing to pay, an economic status of the donor, and an insurance status of the donor.

17. The method of claim 1 wherein said determining is carried out in accordance with costs of testing at least one of the donor, the relative of the donor, and one or more said components of said placenta-umbilical chord complex.

18. The method of claim 1 further comprising:
e) designating a first set of said stored components for private banking with a first business entity; and
f) offering a second set of said stored components for sale to a second business entity, said first set of components being distinct from said second set of components.

19. The method of claim 18 wherein said first entity is a biological matter bank and second entity is one of a cosmetic industry entity and a pharmaceutical industry entity.

20. A method of effecting a business transaction related to a placental-umbilical cord complex of a donor, the placental-umbilical cord complex having a plurality of components, the method comprising:
a) effecting a transaction whereby the donor donates a first set of components of the placental-chord complex;
b) providing to the donor a private banking service for a second set of components of the placental-chord complex,
wherein said first and second sets are distinct.

21. The method of claim 20 wherein said first set of donated components includes cord blood stem cells and the second set of privately banked components includes Wharton's Jelly derived cells.

22. The method of claim 20 wherein said first set of donated components includes Wharton's Jelly derived cells and the second set of privately banked components includes cord blood stem cells.

23. The method of claim 20 wherein said first set of donated components includes at least one generic biomaterial and said second set of privately banked components includes at least one sample of individual undifferentiated or partially differentiated cells.

24. The method of claim 23 wherein said generic biomaterial is selected from the group consisting of vascular tissue, extra-cellular matrix material, cord blood plasma, membranes, enzymes and amniotic fluid.

25. The method of claim 20 wherein a decision of which said components are to be donated and which said components are to be privately banked is carried out in accordance with at least one of donor type information, economic data of the donor, and pricing data of said components of said placenta-umbilical cord complex.

26. The method of claim 25 wherein said pricing data is received via an electronic price data feed.

27. The method of claim 25 wherein mesenchymal stem cells are privately banked if there is an elevated risk of a neurological disease.

28. The method of claim 24 wherein hematopoietic stem cells are privately banked if there is an elevated risk of a cancer.

29. The method of claim 20 further comprising:
c) computing a price of the transaction associated with said donating and said banking.

30. The method of claim 20 further comprising:
c) generating formalized contract text describing a transaction associated with said donating and said banking.

31. A method of determining a price of a financial transaction involving several distinct types of stem cells derived from the placenta-umbilical cord complex of a donor, the method comprising:
a) determining a cost of privately banking first set of samples of stem cells derived from the placenta-umbilical cord complex of the donor;
b) determining a market value of a second set of samples of stem cells derived from the placenta-umbilical cord complex of the donor, said first set and said second set of cells having different stem cell type profile;
c) determining the price of the stem cell transaction by computing a function of said cost of said private banking of said first set of stem cells and said market value of said second set of stem cells.

32. The method of claim 31 wherein said price of said stem cell transaction is further determined in accordance with costs testing at least one of the donor and a family member of the donor.

33. The method of claim 31 further comprising:
d) determining a market value of one or more generic biomaterials derived from the placenta-umbilical cord complex of the donor,
wherein said determining of said price of stem cell transaction is carried out in accordance with said market value of said one or more generic biomaterials.

34. The method of claim 31 wherein said market value of said second set of samples is determined in accordance with at least one of a medical history of the donor, a medical history of a family member of the donor and a genetic profile of the donor.

35. A method of determining a price of a financial transaction involving several distinct components of the placenta-umbilical cord complex of a donor, the method comprising:
a) determining a cost of privately banking first set of components of the placenta-umbilical cord complex of the donor;
b) determining a market value of a second set of components derived from the placenta-umbilical cord complex of the donor, said first set and said second set of components being distinct, said second said of components including a generic biomaterial; and
c) determining the price of the financial transaction by computing a function of said cost of said private banking of said first set of components and said market value of said second set of components.

36. A method of maintaining a computer-based registry different types of undifferentiated or partially differentiated cells derived from different locations in the placenta-umbilical cord complex, the method comprising:
a) creating a new donor record for a potential donor in a placenta-cord complex cells database of the registry;
b) storing donor identification information in the new record;
c) storing sample set identification information in the new record, said sample set including a plurality of samples of distinct stem cell types of the placenta-umbilical cord complex;
d) collecting the sample set from the donor;
e) obtaining donor type information and storing said donor type information in the new record; and
f) storing an availability indication with the new record to indicate which said stem cell types are available for public use;
g) storing the collected sample set in a bank such that individual samples of distinct stem cell types can be obtained from the bank using the stored sample set identification information; and
h) modifying the availability indication for a particular donor record when the availability for public use of at least one type of stem cells changes.

37. A method of utilizing the placenta-umbilical cord complex of a donor, the method comprising:
a) testing for disease at least one of the donor and a relative of the donor; and
b) if results of said testing indicates a disease free state,
i) privately banking stem cells of the placenta-umbilical cord complex; and
ii) offering for sale generic biomaterials of the placenta-umbilical cord complex.

38. A computerized system for determining allocation parameters for a placenta-umbilical chord complex of a donor, the system comprising:
a) a data storage unit adapted to store data about the donor and pricing data about a plurality of placenta chord complex components, said about the donor including economic data and medical-related data;
b) a processing unit for determining a first set of components to be donated and a second set of components to be privately banked,
wherein said determining is carried out in accordance with said pricing data.

39. The method of claim 38 wherein said determining is further carried out in accordance with at least one of said economic data and said medical data of said donor.

40. The system of claim 38 wherein said processing unit is further operative to value a transaction associated with said donating and private banking.

41. The system of claim 39 wherein said processing unit is operative to value said transaction in accordance with prices of testing at least one of the donor, the relative of the donor and a component of said placenta-umbilical chord complex.

42. The system of claim 38 wherein said economic data includes at least one of sum of money the donor is willing to pay, economic status of the donor, and an insurance status of the donor.

43. The system of claim 38 further comprising:
c) a contract generation module for generating formal contract text for the transaction associated with said donating and private banking.

43. A biological material data management system comprising:
a) a data input adapted to receive donor data including an identification of a donor;
b) a transaction logger for logging for each said donor transaction data wherein a plurality of components of placenta-chord complex of the placenta-umbilical cord complex is recovered and transferred; and
c) a data retrieval engine for retrieving a record relating each said component of a respective said placenta-chord complex to a respective said donor.

44. The system of claim 43 further comprising;
d) an inventory tracking system for tracking quantities of a plurality of separately stored components of said placenta-umbilical cord complex.

45. The system of claim 43 further comprising:
e) an on-line sales portal for offering for sale said stored components.

46. The system of claim 45 wherein said on-line sales portal includes
i) a first user interface adapted to receive orders for samples of individual undifferentiated or partially differentiated cells extracted from said placenta-umbilical cord complex, and
ii) a second user interface adapted to receive orders for generic biomaterials derived form said placenta-umbilical cord complex.

47. A computer-readable medium or combination of computer-readable media, containing a program for determining a price of a financial transaction involving several distinct types of stem cells derived from the placenta-umbilical cord complex of a donor, the program comprising code to effect:
a) determining a cost of privately banking first set of samples of stem cells derived from the placenta-umbilical cord complex of the donor;
b) determining a market value of a second set of samples of stem cells derived from the placenta-umbilical cord complex of the donor, said first set and said second set of cells having different stem cell type profile; and
c) determining the price of the stem cell transaction by computing a function of said cost of said private banking of said first set of stem cells and said market value of said second set of stem cells.

48. A computer-readable medium or combination of computer-readable media, containing a program for determining a price of a financial transaction involving several distinct components of the placenta-umbilical cord complex of a donor, the program comprising code to effect:
a) determining a cost of privately banking first set of components of the placenta-umbilical cord complex of the donor;
b) determining a market value of a second set of components derived from the placenta-umbilical cord complex of the donor, said first set and said second set of components being distinct, said second said of components including a generic biomaterial; and
c) determining the price of the financial transaction by computing a function of said cost of said private banking of said first set of components and said market value of said second set of components.

49. A computer-readable medium or combination of computer-readable media, containing a program for maintaining a computer-based registry different types of undifferentiated or partially differentiated cells derived from different locations in the placenta-umbilical cord complex, the program comprising code to effect:
a) creating a new donor record for a potential donor in a placenta-cord complex cells database of the registry;
b) storing donor identification information in the new record;
c) storing sample set identification information in the new record, said sample set including a plurality of samples of distinct stem cell types of the placenta-umbilical cord complex;
d) storing donor type information in the new record; and
e) storing an availability indication with the new record to indicate which said stem cell types are available for public use.

50. The computer-readable medium or combination of computer-readable media as in claim 49 wherein the program further comprises code to effect:
h) modifying the availability indication for a particular donor record when the availability for public use of at least one type of stem cells changes.

51. A device for determining a price of a financial transaction involving several distinct types of stem cells derived from the placenta-umbilical cord complex of a donor, the device comprising:
a) means for determining a cost of privately banking first set of samples of stem cells derived from the placenta-umbilical cord complex of the donor;
b) means for determining a market value of a second set of samples of stem cells derived from the placenta-umbilical cord complex of the donor, said first set and said second set of cells having different stem cell type profile; and
c) means for determining the price of the stem cell transaction by computing a function of said cost of said private banking of said first set of stem cells and said market value of said second set of stem cells.

52. A device for determining a price of a financial transaction involving several distinct components of the placenta-umbilical cord complex of a donor, the device comprising:
a) means for determining a cost of privately banking first set of components of the placenta-umbilical cord complex of the donor;
b) means for determining a market value of a second set of components derived from the placenta-umbilical cord complex of the donor, said first set and said second set of components being distinct, said second said of components including a generic biomaterial; and
c) means for determining the price of the financial transaction by computing a function of said cost of said private banking of said first set of components and said market value of said second set of components.

53. A device for maintaining a computer-based registry different types of undifferentiated or partially differentiated cells derived from different locations in the placenta-umbilical cord complex, the device comprising:
a) means for creating a new donor record for a potential donor in a placenta-cord complex cells database of the registry;
b) means for storing donor identification information in the new record;
c) means for storing sample set identification information in the new record, said sample set including a plurality of samples of distinct stem cell types of the placenta-umbilical cord complex;
d) means for storing donor type information in the new record; and
e) means for storing an availability indication with the new record to indicate which said stem cell types are available for public use.

54. The device of claim 53 further comprising:
h) means for modifying the availability indication for a particular donor record when the availability for public use of at least one type of stem cells changes.
